(19) United States
(12) Patent Application Publication
(10) Pub. No.: US 2006/0155293 A1
(43) Pub. Date: Jul. 13, 2006

(54) External rotation cut guide

(75) Inventors: Shawn E. McGinley, Fort Wayne, IN (US); James E. Grimm, Winona Lake, IN (US); Maleata Hall, Warsaw, IN (US); Sudip Hui, Warsaw, IN (US)

Correspondence Address: GREER, BURNS & CRAIN, LTD.;ATTN: JAMES K. FOLKER, 300 SOUTH WACKER DRIVE, SUITE 2500, CHICAGO, IL, 60606, US

(73) Assignee: ZIMMER TECHNOLOGY

(21) Appl. No.: 11/030,805

(22) Filed: Jan. 7, 2005

(51) Int. Cl. A61F 5/00

(52) U.S. Cl. 606/87

(57) ABSTRACT

The present invention relates to a bone-cutting guide intended to be temporarily positioned against a resected bone surface at a bone-cutting surface. The bone-cutting guide is also positioned at at least one secondary bone surface, and configured to receive a cutting member to cut the bone at the bone-cutting surface. The bone-cutting guide includes a tower and a paddle extending from the tower. The paddle is configured to be positioned at the bone-cutting surface, and has a cutting tool receiving portion configured and arranged to receive the cutting member to cut the bone at the bone-cutting surface. The bone-cutting guide also includes a base extending from the tower and configured to be positioned on the resected bone surface. The base has at least one bone-engaging surface configured and arranged to be positioned at the at least one secondary bone surface. An angle adjuster is provided for adjusting the angle between the tower and the base in the plane of the resected bone surface.

The present invention relates generally to a bone-cutting guide used during knee arthroplasty, where the bone-cutting guide is used for guiding a cutting member during cutting of the femur. More particularly, the present invention relates to a bone-cutting guide configured to make an external rotation cut of a femur. The concept of the present invention can be applied to many different types of arthroplasty, such as, for example, Unicompartmental Knee Arthroplasty (UKA) and Total Knee Arthroplasty (TKA).

Throughout this application various positional terms—such as distal, proximal, medial, lateral, anterior and posterior—will be used in the customary manner when referring to the human anatomy. More specifically, “distal” refers to the area away from the point of attachment to the body, while “proximal” refers to the area near the point of attachment to the body. For example, the proximal femur refers to the portion of the femur near the hip, while the distal femur refers to the portion of the femur near the tibia. The terms “medial” and “lateral” are also essentially opposites, where “medial” refers to something situated closer to the middle of the body, while “lateral” refers to something situated closer to the left side or the right side of the body (than to the middle of the body). Finally, with regard to anterior and posterior, “anterior” refers to something situated closer to the front of the body and “posterior” refers to something situated closer to the rear of the body. As shown in FIG. 1, the medial direction is indicated by the letter “m”, the lateral direction is indicated by the letter “l”, the anterior direction is indicated by the letter “a”, and the posterior direction is indicated by the “p”.

The present invention provides an alternative approach to known methods and devices used for guiding the cutting blade for cutting the distal femur during knee arthroplasty. After resection of the distal femur, there may be a need to make an anterior rough cut to create a flat plane. This plane can then be easily referenced by subsequent instruments, and can eliminate problems encountered by the surgeon involving the degree-of-freedom available for a particular instrument. Following the rough cut, the femoral finishing cut guide can be used.

The present invention provides a minimally invasive and relatively uncomplicated tool that can be used to accurately make an anterior rough cut. Further, the present invention provides a cutting tool with a cutting guide that can be adjusted in both the anterior and posterior directions, as well as the internal and external directions.

### SUMMARY OF THE INVENTION

The present invention relates to a bone-cutting guide intended to be temporarily positioned against a resected bone surface at a bone-cutting surface. The bone-cutting guide is also positioned at at least one secondary bone surface, and configured to receive a cutting member to cut the bone at the bone-cutting surface. The bone-cutting guide includes a tower and a paddle extending from the tower. The paddle is configured to be positioned at the bone-cutting surface, and has a cutting tool receiving portion configured and arranged to receive the cutting member to cut the bone at the bone-cutting surface. The bone-cutting guide also includes a base extending from the tower and configured to be positioned on the resected bone surface. The base has at least one bone-engaging surface configured and arranged to be positioned at the at least one secondary bone surface. An angle adjuster is provided for adjusting the angle between the tower and the base in the plane of the resected bone surface.

More specifically, the present invention provides a bone-cutting guide intended to be used during knee surgery after a distal end of a femur is cut, for positioning a cutting member into proper orientation for cutting a portion of the femur. The bone-cutting guide includes a base configured to be positioned on the femur in generally the same plane as the distal cut, and a tower extending from the base in generally the same plane as the distal cut. Also, the bone-cutting guide includes a paddle extending from the tower having a cutting tool receiving portion forming a cutting plane generally orthogonal to the distal cut. The cutting tool receiving portion is configured and arranged to receive the cutting member. The tower is pivotable about a pivot point on the base to rotate the tower either internally or externally.

Additionally, the present invention relates to a bone-cutting guide intended to be temporarily positioned against a distal cut surface of a femur to receive a cutting member to cut an anterior rough cut. The bone-cutting guide includes a tower and a paddle extending from the tower and having a first contact surface and a second contact surface configured and arranged to contact the distal cut surface one at a time. A cutting tool receiving portion extends from the first contact surface to the second contact surface and is configured and arranged to receive the cutting member. The paddle also has an image probe for tracking the location of any point on the bone-cutting guide with respect to a patient. The bone-cutting guide also includes a base extending from said tower for being positioned generally flush on the distal cut surface. The base has at least one bone-engaging surface configured and arranged to be positioned against a posterior side surface of the femur. The tower is pivotable about a pivot point on the base.

Another feature of the present invention relates to a connector for pivotally connecting a tower to a base for positioning a cutting member associated with the tower into proper orientation for cutting a bone. The connector includes a pivot member pivotally connecting an interior portion of the tower to the base, and an engaging member configured for selectively imparting a force on the tower at the interior portion. A receiving portion is disposed on said interior portion of the tower and is configured for receiving the engaging member, where the engaging member is configured for pivoting the tower about the pivot member when the force is imparted.

### BRIEF DESCRIPTION OF THE SEVERAL VIEWS OF THE DRAWINGS

Preferred embodiments of the present invention are described herein with reference to the drawings wherein:

FIG. 1 is a top perspective view of a bone-cutting guide of the present invention disposed on a distal cut surface of a femur;

FIG. 2 is an exploded view of the bone-cutting guide of FIG. 1;

FIG. 3 is a partial perspective view of a tower of the bone-cutting guide of FIG. 1; and

FIG. 4 is a partially cut-away perspective view of a connection between the tower and the base of the bone-cutting guide of FIG. 1.

### DETAILED DESCRIPTION OF THE INVENTION

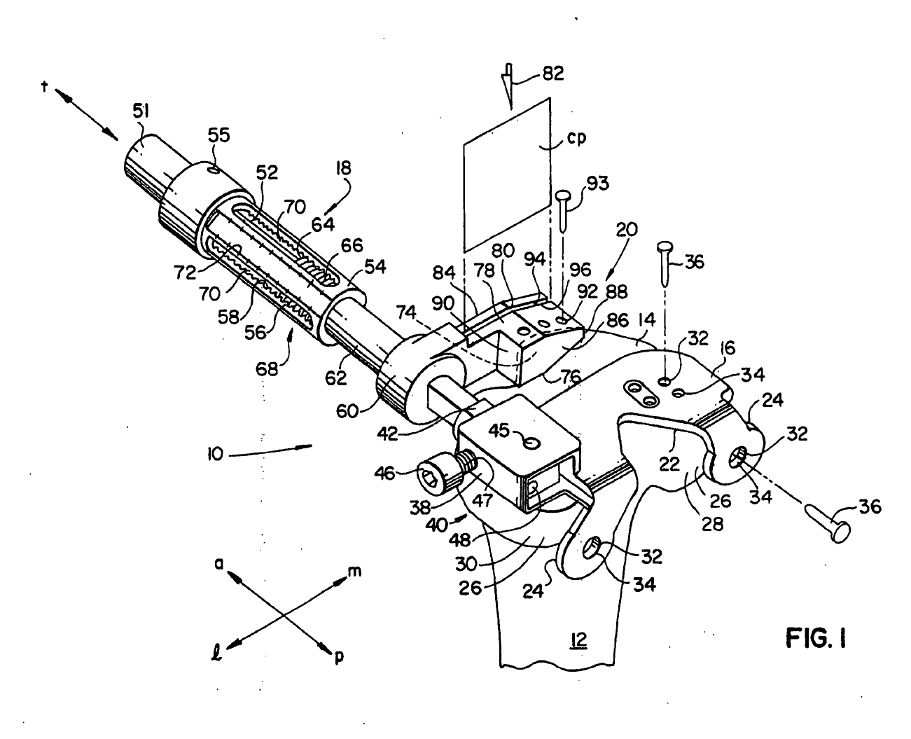

Turning to FIG. 1, one embodiment of the present bone-cutting guide 10 will be shown and described, with FIG. 1 showing the bone-cutting guide in position on a femur 12. The bone-cutting guide 10 is configured and arranged to be temporarily positioned upon a resected distal surface 14 of the femur 12 for making a rough anterior cut generally perpendicular to the resected surface 14. Although the present bone-cutting guide 10 is shown and described with respect to knee surgery, and specifically with respect to the cutting of the femur, it is contemplated that the bone-cutting guide can be used for other bone cutting procedures, such as a posterior cut.

Knee arthroplasty is the rebuilding of the knee, which can be done by resurfacing or relining the ends of bones where cartilage has worn away and bone has been destroyed. Arthroplasty also refers to total joint replacement, where all or part of an arthritic joint is removed and replaced with metal, ceramic, and/or plastic parts. Resection is the removal of part or all of a bone, which is often done to improve function and relieve pain in the knee. Resection is performed by a surgeon, and an anterior rough cut is made generally perpendicular to the resection surface 14 to provide a flat plane that can be easily referenced by subsequent instruments, and that can provide the surgeon with room to manipulate the instruments.

The bone-cutting guide 10 preferably includes three main components: a base 16, a tower 18, and a paddle 20. The base 16 is generally thin and elongate, having a planar contact surface 22 configured and arranged to be positioned flush on the resected surface 14 of the femur 12. When the base 16 is engaged on the posterior side of the distal surface 14 of the femur 12, the base preferably extends substantially from the medial to the lateral side. In the preferred embodiment, the base 14 has an extension length to accommodate the smallest and largest patient sizes, but any length is contemplated. Further, while the preferred embodiment is thin and elongate, any shaped base 14 which has a planar contact surface 22 configured to be positioned on the resected surface 14 is contemplated.

Extending generally perpendicular from the base 16 are at least one, but preferably two, bone-engaging surfaces 24 that are configured and arranged to be positioned at at least one secondary bone surface 26. The secondary bone surface 26 is preferably a posterior side surface of the femur, and the bone-engaging surfaces 24 preferably engage the medial condyle 28 and a lateral condyle 30. While the base of FIG. 1 is side specific in that it is configured and arranged to engage the medial and lateral condyles 28, 30, it is contemplated that the bone-engaging surfaces 24 can be side specific to other sides of other bones such as by being contoured to the secondary bone surface 26, or can be universal for any secondary bone surface. Additionally, more than two bone-engaging surfaces 24 can also be included, if desired.

FIG. 1 shows a perspective view of the base 16 having a base attachment arrangement 32 configured for attaching the base 16 of the bone-cutting guide 10 to the femur 12. Preferably, the base attachment arrangement 32 includes at least one but preferably a plurality of apertures 34 provided on the base 16. The aperture 34 is used with a fastener such as pin 36. In use, the pin 36 is inserted through the aperture 34 and extends into the femur 12 to secure the bone-cutting guide 10 in position. In the preferred embodiment, two pins 36 are inserted through the apertures 34 in the bone-engaging surfaces 24 into the medial and lateral condyles 28, 30, and four pins are inserted into the resected surface 14, but it is also contemplated that other base attachment arrangements 32 could be used to attach the bone-cutting guide 10 to the bone, such as protrusions on the surface of the base configured to be embedded in the bone, or by applying adhesive to the base. Alternatively, the base 16 can be held in place by the surgeon.

In the embodiment shown in FIG. 1, the tower 18 extends generally perpendicularly from the base 16 in generally the same plane as the resected surface 14 of the femur 12 in the anterior direction. Further, the tower housing 38 houses a connector 40, and is preferably integral with the base 16. Preferably an elongate member such as a rod or cylinder, the tower 18 preferably has an interior portion 42 which is preferably pivotally connected to the housing 38 and to the base 16 with a pivot member 44. Pivoting of the tower 18 occurs about a pivot point 45.

When connected to the base 16, the tower 18 preferably forms a general “L” configuration. An angle adjuster 46 associated with the tower 18, the base 16, and the pivot point 45 pivots the tower with respect to the base, adjusting the angle of the “L” either acutely or obtusely. This configuration of angle adjustment is known as internal/external adjustment or rotation. When an instrument, such as the cutting guide 10, is rotated laterally away from a perpendicular line that intersects a line formed between the medial condyle 28 and the lateral condyle 30, the instrument is externally rotated. External angle adjustment makes the angle of the “L” obtuse, while internal angle adjustment makes the angle of the “L” acute.

Referring now to FIG. 4, the connector 40 is configured to pivot the interior portion 42 of the tower 18 about the pivot member 44 at the pivot point 45. The pivot member 44 pivotally connects the tower 18 to the base 16, while an engaging member 47 selectively imparts a force on the interior portion 42 a distance from the pivot point 45 to effect pivoting of the tower. The interior portion 42 of the tower 18 includes a receiving portion 48 configured for receiving the engaging member 47. Preferably a “U”-slot along the longitudinal axis “t” of the tower 18, the receiving portion 48 preferably positively retains a head 49 of the engaging member 47 within the “U”-slot.

When the engaging member 47 imparts a force on the interior portion 42, the tower 18 is pivoted about the pivot point 45. In the preferred embodiment, the angle adjuster 46 is preferably a threaded knob extending through an opening in the tower housing 38 and includes the engaging member 47. When the angle adjuster 46 is adjusted, the engaging member 47 imparts a force on the interior portion 42 either towards or away from the tower 18 depending on the direction of threading and the direction that the knob is rotated. Additionally, in the preferred embodiment, a biasing member 50 is disposed to counter the force imparted by the engaging member 47. The biasing member 50 is preferably a simple spring attached to the tower housing 38 and is configured to abut the interior portion 42 opposite the receiving portion 48.

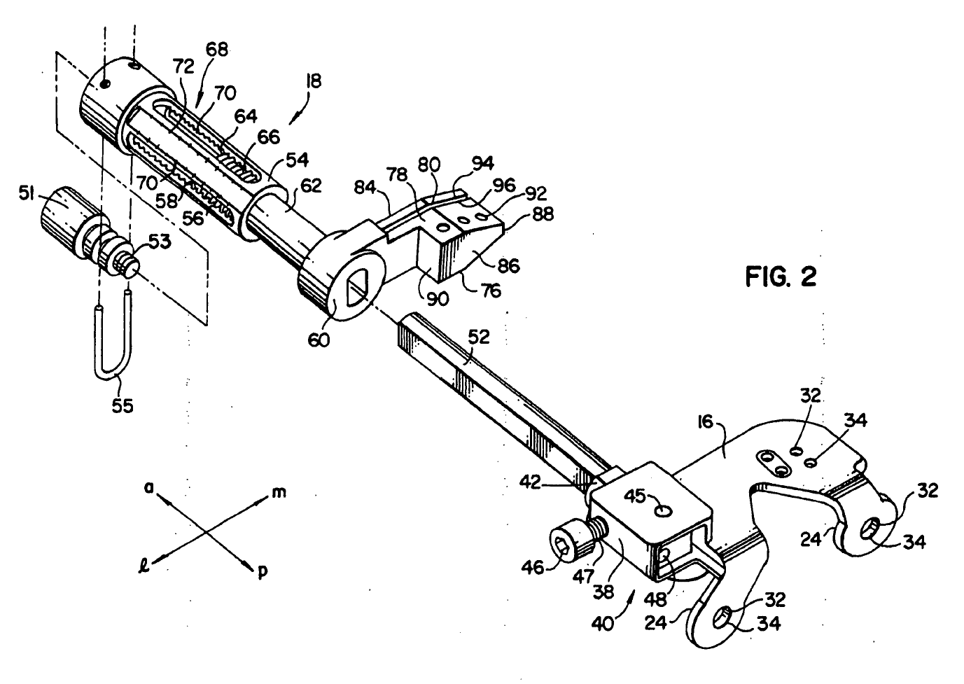

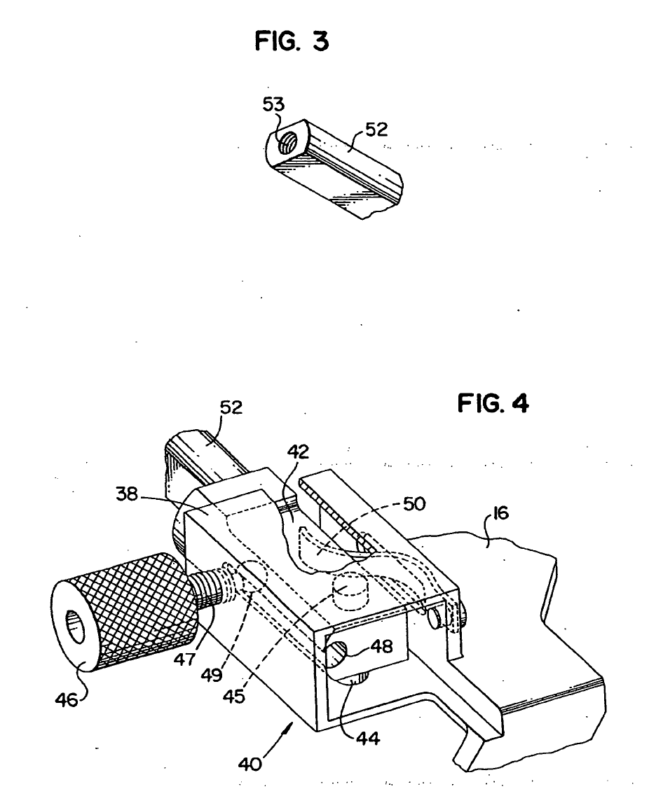

Referring now to FIGS. 1 and 2, while the tower 18 is configured to pivot about the pivot point 45, a portion of the tower is preferably configured to rotate about the longitudinal axis “t” of the tower. In particular, the tower 18 includes an exterior portion 51 opposite the pivot point 45 which is non-rotatable with respect to the interior portion 42. The exterior portion 51 and the interior portion 42 are connected to each other by a center portion 52 of the tower 18. Preferably, the exterior portion 51 and the center portion 52 are statically and removably connected to each other, such as with corresponding threading 53 (FIGS. 2 and 3). Together, the interior portion 42, the exterior portion 51 and the center portion 52 are preferably configured to be static with respect to each other.

The tower 18 also preferably includes a casing 54 circumferentially disposed around the center portion 52, and fastened to the exterior portion 51 with a fastener 55. An interior surface 56 of the casing preferably has a threaded portion 58 disposed thereon. The casing 54 and the center portion 52 are operatively attached to each other at the exterior portion 51. In this configuration, the casing 54 preferably rotates about the longitudinal axis “t” of the tower 18 with respect to the interior portion 42, the exterior portion 51 and the center portion 52.

In the preferred embodiment and referring to FIG. 2, portions of the tower 18 are assembled as a unitary piece with the base 16, including the interior portion 42 and the center portion 52. Other portions of the tower 18 are assembled as a unitary piece with the paddle 20, including the exterior portion 51 and the casing 54. In this configuration, the paddle 20, the casing 54 and the exterior portion 51 can be selectively removed from or assembled onto the tower center portion 52, and secured into place with the corresponding threading 53 on the center portion (FIG. 3) and the exterior portion, for example.

Referring back to FIG. 1, the paddle 20 extends generally perpendicularly to the tower 18. Having a configuration preventing the rotation of the paddle 20 about the tower 18, the paddle is preferably disposed on a paddle sleeve 60. In the preferred embodiment, the paddle sleeve 60 has a non-uniform diameter to prevent the paddle 20 from rotating about the tower 18. A middle sleeve 62 is preferably integral with and adjacent to the paddle sleeve 60. Configured to displace with the paddle sleeve 60, the middle sleeve 62 has an external end 64 having a threaded portion 66 configured to engage the threaded portion 58 of the casing 54. Preferably, the paddle sleeve 60 and the middle sleeve 62 are integral.

When the casing 54 is rotated about the longitudinal axis “t” of the tower 18, the middle sleeve 62 and the paddle 20 are displaced or translated along the length of the tower in the anterior and the posterior directions, while the exterior portion 51, the interior portion 42, and the center portion 52 remain static. Since the center portion 52 and the paddle sleeve 60 are configured to prevent rotation of the paddle 20 about the tower 18, the resulting forces that act on the threaded portion 66 of the middle sleeve 62 by the tower threaded portion 58 tend to push or pull the sleeve towards or away from the base 16, depending on the direction of rotation and the direction of threading. In this manner, the anterior/posterior location of the paddle 20 can be adjusted to a desired position along the length of the tower 18. Further, the interior portion 42 of the tower is preferably slightly larger than the center portion 52, forming a stop to prevent excessive posterior translation of the paddle 20.

The casing 54 preferably includes an indicator 68, which in this embodiment includes a plurality of windows 70 which permit the surgeon to see the relative location of the middle sleeve 62 with respect to the casing 54. This information, in turn, can be used to determine the anterior/posterior translation of the paddle. A scale 72 may be disposed on the indicator 68 to allow a precise measurement of the location of the paddle 20 by aligning the end of the threaded sleeve 66 to the scale, which may be calibrated with other instruments used during a procedure.

The paddle 20 is configured to be positioned distal of the femur 12 adjacent a bone-cutting surface 74 (shown hidden), and has a first contact surface 76 generally opposed to a second contact surface 78, forming, when viewed towards the anterior direction, a generally “A”-shaped configuration, although other shapes are contemplated. The first contact surface 76 and the second contact surface 78 are configured to selectively contact the resection surface 14. A cutting tool receiving portion 80 extends from the first contact surface 70 to the second contact surface 78, generally perpendicular to both surfaces, and is configured for receiving a cutting member 82. Preferably, the cutting tool receiving portion 80 includes a slot in the paddle 20 that is configured to receive, restrict and guide the cutting member 82, such as a blade, to cut the bone within the area of the slot at the bone-cutting surface 74. Further, the cutting member 82 is preferably attached to a reciprocating or oscillating saw (not shown), or other cutting device configured for use during knee arthroplasty, or any other bone cut.

The cutting tool receiving portion 80 is preferably located between an anterior surface 84 and a posterior surface 86 of the paddle 20, and further, preferably extends to an outer surface 88 of the paddle, opposite a tower-facing surface 90. Further, it is contemplated that a plurality of cutting tool receiving portions 80 may be disposed on the paddle 20. For example, the paddle 20 can have a plurality of cutting tool receiving portions 80 in parallel, as well as in oblique orientation to each other.

In the preferred embodiment, the cutting tool receiving portion 80 defines a cutting plane “cp” that is generally orthogonal to said first and second contact surfaces 76, 78; is generally perpendicular to the resected surface 14 of the femur 12; and is generally parallel to the at least one secondary bone surface 26. However, the cutting plane “cp” can be oriented to be non-orthogonal to the resected surface 14, if desired.

Further, as was described with respect to the tower 18, the paddle 20 can be adjusted in various directions to position the cutting plane “cp” in the desired location. When the first or second contacting surface 76, 78 is positioned on the resected bone in a generally flush engagement, the base 16 references the resection surface 14 to position the paddle 20 and the cutting plane “cp” into the desired location at the bone cutting surface 74.

For example, if the surgeon wants the cutting plane “cp” to be moved in either one of the anterior or posterior directions, the casing 54 is rotated with respect to the middle sleeve 62, and the threaded portion 58 forces the paddle 20 to displace along the length of the tower 18 towards or away from the base 16, depending on the direction of rotation and the direction of threading. If the surgeon wants to adjust the cutting plane “cp” in one of the medial or lateral directions, the tower 18 can be pivoted about the pivot point 45 using the angle adjuster 46.

The paddle 20 preferably includes a paddle attachment arrangement 92, similar to the base attachment arrangement 32, configured to attach the paddle 20 to the resected surface 14 of the distal cut, preferably with a fastener such as a pin 93. Alternatively, the paddle 20 can attached to the bone in other ways, or can be held in place by the surgeon. When in the desired position, the cutting member 82 is inserted into the cutting tool receiving portion 80, and guided along at least one, but preferably a plurality of guide surfaces 94 in the cutting tool receiving portion, and an anterior rough cut is made generally perpendicular with the resected surface 14.

In certain instances, the surgeon may want to make the anterior rough cut generally acute or obtuse to the resected surface 14, as opposed to the orthogonal orientation of the cutting plane shown in FIG. 1. In that instance, it is contemplated that the cutting plane “cp” of the cutting tool receiving portion 80 can be oblique to the plane of the paddle 20, or that the paddle can be configured to pivot with respect to the paddle sleeve 60. In the preferred embodiment including the slot as the cutting tool receiving portion 80, the oblique cutting plane “cp” would be effected by angling the slot within the paddle 20. An angled cutting tool receiving portion on a bone-cutting guide 10 can be used by a surgeon to alter the femoral slope, for example.

In addition, the cutting tool receiving portion 80 can be non-linear when a curved surface is required. Further, the cutting tool receiving portion 80 can have any orientation or alignment relative to the paddle 20. Although the cutting tool receiving portion 80 is preferably a slot, it is also contemplated that other receiving portions can be incorporated. For example, the anterior surface or the posterior surface of the paddle 20 can form the guide surface 94.

In the preferred embodiment, an image guide probe 96 is configured and arranged to track the location of any point on the bone-cutting guide 10, and preferably, the location of the cutting tool receiving portion 80, with respect to the patient. Using an image guidance system 98, the location of the surgical instrumentation can be tracked, preferably in real time, when the instrument enters the field relative to the patient, and can be displayed on a computer.

Examples of various computer assisted navigation systems which are known in the art are described in U.S. Pat. Nos. 5,682,886; 5,921,992; 6,096,050; 6,348,058 B1; 6,434,507 B1; 6,450,978 B1; 6,490,467 B1; 6,491,699 B1; and U.S. patent application Ser. Nos. 10/357,592 and 10/794,657, the disclosure of each of these patents is hereby incorporated herein by reference. Image guidance techniques typically involve acquiring preoperative images of the relevant anatomical structures and generating a data base which represents a three dimensional model of the anatomical structures. The relevant surgical instruments typically have a known and fixed geometry which is also defined preoperatively. During the surgical procedure, the position of the instrument being used is registered with the anatomical coordinate system and a graphical display showing the relative positions of the tool and anatomical structure may be computed in real time and displayed for the surgeon to assist the surgeon in properly positioning and manipulating the surgical instrument with respect to the relevant anatomical structure. Such techniques typically include, but are not limited to tracking technologies, such as optical, electro-magnetic and gyroscopic, and imaging technologies, such as fluoroscopic, computed tomography and magnetic resonance imaging, for example.

In the preferred bone-cutting guide 10, the image guide probe 96 is disposed in the cutting tool receiving portion 80, and used with an image guidance system to track the internal or external rotation of the tower 18 with respect to the base 16. Further, using image guidance, the user can adjust and track the internal/external angle a precise amount, preferably in finite increments of angles, using the angle adjuster 46. This feature is of particular use during a procedure where the location of the cut must be determined to a small degree of error, or when an increment of an angle is required, such as when other instruments must reference the location of the cut.

Preferably provided with the bone-cutting guide 10 is a second base which is preferably a mirror image of the base 16. The preferred unitary assembly of the paddle 20, (with the middle sleeve 62, the casing 54 and the exterior portion 51) is configured to be removable from the unitary assembly of the base 16. The paddle 20 and the accompanying assembly can be removed from the base 16 and attached to the second base which is the mirror image of base 16. While the base 16 is configured for cutting the lateral compartment of the right leg (RTL) and the medial compartment of the left leg (LTM), the mirror image of base 16 is configured for the lateral compartment of the left leg (LTL) and the medial compartment of the right leg (RTM).

Specifically, in FIG. 1, the bone-cutting guide 10 is engaged on the distal femur 12 with the base 16 disposed on the posterior side and the paddle 20 positioned above the resected surface 14 at the medial, anterior side of a left leg (LTM). In this configuration, the first contact surface 76 is configured to engage the resected surface 14 and the paddle 20 is configured to extend generally parallel to and in the same direction as the base 16. However, if the exterior portion 51 is unfastened from the center portion 52, and if the paddle 20 and the associated assembly are removed from the center portion 52, the paddle 20 can be reattached to the mirror image base. The paddle 20 and the associated assembly are attached to the mirror image base in the same manner as it is attached to the base 16, except the paddle and the associated assembly are flipped over so that the second contact surface 78 is configured to engage the resected surface 14. In this configuration, the paddle 20 makes an “L”-shape with the tower 18 which extends parallel to the “L”-shape made between the tower and the mirror image base. Thus, the same paddle 20 (and associated assembly) can be used with multiple bases for use on both the medial and lateral compartments of the knee, as well as the left and the right knees, or any other type of bone cut.

While various embodiments of the present invention have been shown and described, it should be understood that other modifications, substitutions and alternatives may be apparent to one of ordinary skill in the art. Such modifications, substitutions and alternatives can be made without departing from the spirit and scope of the invention, which should be determined from the appended claims.

Various features of the invention are set forth in the appended claims.

What is claimed is:

1. A bone-cutting guide intended to be temporarily positioned against a resected bone surface at a bone-cutting surface and at at least one secondary bone surface, and to receive a cutting member to cut the bone at the bone-cutting surface, the bone-cutting guide comprising:
a tower;
a paddle extending from said tower configured to be positioned at the bone-cutting surface, and having a cutting tool receiving portion configured and arranged to receive the cutting member to cut the bone at the bone-cutting surface;
a base extending from said tower and configured to be positioned on the resected bone surface having at least one bone-engaging surface configured and arranged to be positioned at the at least one secondary bone surface; and
an angle adjuster for adjusting the angle between said tower and said base in the plane of the resected bone surface.

2. The bone-cutting guide according to claim 1, wherein said paddle has a first contact surface generally opposed to a second contact surface, and said cutting tool receiving portion extends from said first contact surface to said second contact surface.

3. The bone-cutting guide according to claim 2, wherein said cutting tool receiving portion forms a cutting plane that is generally orthogonal to said first and second contact surfaces.

4. The bone-cutting guide according to claim 1, further comprising an image guide probe configured and arranged to track the location of the bone-cutting guide with respect to a patient.

5. The bone-cutting guide according to claim 1, wherein said paddle is prevented from rotation about the tower and is configured to extend generally parallel to the base.

6. The bone-cutting guide according to claim 1, wherein said paddle is configured to be displaced along the length of said tower.

7. The bone-cutting guide according to claim 1, wherein said tower is configured to pivot about a pivot point associated with said base.

8. The bone-cutting guide according to claim 1 further comprising a base attachment arrangement on said base configured to attach the bone-cutting guide to the bone.

9. The bone-cutting guide according to claim 1, wherein said base is configured to be positioned flushly on a distal cut of a femur, and wherein the bone-cutting surface is an anterior location on said distal cut, and wherein said paddle is constructed and arranged to be positioned over said distal cut such that said cutting tool receiving portion is located at the bone-cutting surface.

10. The bone-cutting guide according to claim 2, wherein said base further comprises two bases, said bases being mirror images, wherein said paddle is configured to be attacheable to one of said bases and to engage the resected bone surface with said first contact surface, and wherein said paddle is also configured to be attacheable to said other base and to engage the resected bone surface with said second contact surface.

11. A bone-cutting guide intended to be used during knee surgery after a distal end of a femur is cut, for positioning a cutting member into proper orientation for cutting a portion of the femur, said cutting guide comprising:
a base configured to be positioned on the femur in generally the same plane as the distal cut;
a tower extending from said base in generally the same plane as the distal cut, wherein said tower is pivotable about a pivot point on said base to rotate the tower one of internally and externally, wherein said pivoting is effected by an angle adjuster configured to impart a force on said tower; and
a paddle extending from said tower having a cutting tool receiving portion forming a cutting plane generally orthogonal to the distal cut, said cutting tool receiving portion configured and arranged to receive the cutting member.

12. The bone-cutting guide of claim 11 wherein said paddle is disposed on a threaded sleeve associated with said tower, wherein said paddle is prevented from rotation about the tower.

13. The bone-cutting guide of claim 12 further comprising a threaded casing circumferentially disposed about the tower and configured to rotate about the tower, wherein said threaded casing and said threaded sleeve are operatively engaged and wherein said paddle is displaced by the relative rotation of said casing to said tower.

14. A bone-cutting guide intended to be temporarily positioned against a distal cut surface of a femur to receive a cutting member to cut an anterior rough cut, the bone-cutting guide comprising:
a tower;
a paddle extending from said tower having a first contact surface and a second contact surface configured and arranged to selectively contact the distal cut surface, and a cutting tool receiving portion extending from said first contact surface to said second contact surface configured and arranged to receive the cutting member, said paddle having an image probe for tracking the location of any point on said bone-cutting guide with respect to a patient; and
a base extending from said tower and configured for being positioned generally flush on the distal cut surface, said base having at least one bone-engaging surface configured and arranged to be positioned against a posterior side surface of the femur;
wherein said tower is pivotable about a pivot point on said base.

15. The bone-cutting guide of claim 14 wherein said image probe is used as part of an image guidance system configured to track one of the internal and external rotation of said tower with respect to said base.

16. The bone-cutting guide of claim 15 further comprising an angle adjuster wherein said image guidance system allows the user to manipulate said angle adjuster to finitely track said one of internal and external rotation of said tower to a desired location.

17. A connector pivotally connecting a tower to a base for positioning a cutting member associated with the tower into proper orientation for cutting a bone, said connector comprising:
a pivot member pivotally connecting an interior portion of the tower to the base;
an engaging member configured for selectively imparting a force on the tower at said interior portion; and
a receiving portion disposed on said interior portion configured for receiving said engaging member;
wherein said engaging member is configured for pivoting the tower about said pivot member when said force is imparted.

18. The connector of claim 17 wherein said receiving portion further comprises a “U”-slot and said engaging portion further comprises a head, wherein said head is positively retained within said “U”-slot.

19. The connector of claim 17 wherein a biasing member is configured for imparting a biasing force on the tower to counter said force imparted by said engaging member.

20. The connector of claim 17 further comprising an angle adjuster associated with said engaging member for selectively imparting said force on the tower.
